(19) United States
(12) Patent Application Publication
(10) Pub. No.: US 2005/0137549 A1
(43) Pub. Date: Jun. 23, 2005

(54) Use of swirl-like adhesive patterns in the formation of absorbent articles

(75) Inventors: Jeffrey Dean Lindsay, Appleton, WI (US); Fung-jou Chen, Appleton, WI (US)

Correspondence Address: DORITY & MANNING, P.A., POST OFFICE BOX 1449, GREENVILLE, SC, 29602-1449, US

(73) Assignee: Kimberly-Clark Worldwide, Inc.

(21) Appl. No.: 10/743,259

(22) Filed: Dec. 22, 2003

(51) Int. Cl. A61F 13/15; A61F 13/20

(52) U.S. Cl. 604/385.01

(57) ABSTRACT

Absorbent products are disclosed comprised of multiple components. At least two of the components are adhered together using an adhesive. In accordance with the present invention, the adhesive is applied in between the components according to a non-uniform pattern that varies as a function of distance. For example, the adhesive pattern may change according to at least one of pattern breadth or adhesive dose in weight per unit area. In one particular embodiment, the pattern contains a swirl-like pattern containing a plurality of loops. Over a particular distance, a change may occur in the size of the loops, in the density of the loops, and/or in alternating between loops and a linear bead. In this manner, placement of the adhesive is carefully controlled in order to counteract mechanical stresses that are placed on the absorbent product during use.

### BACKGROUND OF THE INVENTION

Disposable absorbent products currently find widespread use in many applications. For example, in the infant and childcare areas, diapers and training pants have generally replaced reusable cloth absorbent articles. Other typical disposable absorbent products include feminine care products such as sanitary napkins or tampons, adult incontinence products, and healthcare products such as surgical drapes or wound dressings. A typical disposable absorbent product generally comprises a composite structure including a covering, a liner, and an absorbent structure between the covering and the liner. The disposable absorbent products, when appropriate, also may include some type of fastening system for fitting the product onto a wearer. Adhesives are generally used to join the different parts of the disposable absorbent product together. The adhesives are typically applied to the components using one or more nozzles that deliver the adhesive as a linear bead, in a swirl pattern, as random filaments (e.g., meltblown techniques in which turbulent air entrains extruded filaments of adhesive), or as a spray. Controlled delivery of adhesives in swirl patterns or linear beads can be particularly important for some applications.

To ensure secure attachment between the components, while using an economical quantity of adhesive and producing an acceptable visual appearance, the adhesive should be accurately positioned on one of the components according to carefully controlled amounts. For example, it may be desirable to vary the pattern and/or dose of the adhesive with position. The mechanical stresses which must be resisted by an adhesive in a product are rarely uniform and can vary significantly with position in the article. Thus, greater amounts of adhesive may be necessary where the mechanical stresses are at a maximum.

Unfortunately, adhesive nozzles that have been used in the past have been substantially static such that the nozzles were incapable of varying the pattern and/or amount of adhesive during operation. Thus, a need currently exists for a process for applying adhesives to components in the manufacture of absorbent products in which the pattern by which the adhesive is applied and/or the amount of adhesive per area that is applied can be varied rapidly and within desired areas. A need also exists for improved absorbent products made according to the above method.

### SUMMARY OF THE INVENTION

In general, the present invention is directed to a method for applying adhesives to components during the automated construction of a disposable absorbent product. The present invention is also directed to the products produced by the method of the present invention. Such products may be manufactured on an automated machine at industrially practical rates, such as a rate of about 5 articles per minute or greater, or about 50 articles per minute or greater, or about 500 articles per minute or greater. According to the present invention, an adhesive is applied in between a pair of opposing components according to a non-uniform pattern that varies as a function of distance. In this manner, controlled amounts of adhesive may be applied to the components in order to improve the overall properties of the product. For instance, the amount of adhesive applied to the components may be varied in order to counteract the mechanical stresses to which the components undergo during use.

As used herein, the term “adhesive” is intended to mean a substance that is capable of bonding other substances together by surface attachment. Adhesives useful in the present invention may generally be of any known type, such as a thermoplastic hot-melt adhesive, a reactive adhesive, a pressure sensitive adhesive, a UV curable adhesive, silicone-based adhesives, proteinaceous adhesives, thermosetting adhesives, and the like. One example, for instance, of a thermoplastic hot-melt adhesive includes a synthetic, olefin-based adhesive with a micro-crystalline wax, available from National Starch and Chemical Company under the trade designation 70-4741. An example of a reactive adhesive includes crosslinked amine-epoxide compounds and moisture-cured polyurethanes.

In one particular embodiment of the present invention, an absorbent product is formed comprising multiple components. The components can include, for instance, a liner, an outer cover, and an absorbent structure positioned between the liner and the outer cover. In accordance with the present invention, an adhesive is positioned between at least two of the components. The adhesive may be applied, for instance, at least partly according to a swirl-like pattern. The adhesive pattern changes as a function of distance. More particularly, the adhesive pattern changes according to at least one of pattern breadth or adhesive dose in weight per unit area in a particular direction (i.e., the direction of application in the article, defined by the path of the article relative to the adhesive applicator, or the path of the adhesive applicator relative to the article). Alternatively, the adhesive may be delivered as a spray or as random filaments in which the pattern breadth or adhesive dose in weight per unit area is controlled during delivery to vary along a particular direction.

For example, in one embodiment, a swirl-like pattern may comprise a plurality of loops having a size that changes as a function of distance. In another embodiment, a swirl-like pattern comprises a plurality of loops that has a density in loops per distance that changes as a function of distance. In still another embodiment, the adhesive pattern alternates between a swirl-like pattern and a continuous bead. For many applications, the adhesive pattern is continuous, although in some circumstances the pattern may be discontinuous containing areas where no adhesive is applied.

The components adhered together according to the present invention may vary depending upon the particular product being formed. For instance, in one embodiment, the process of the present invention may be used to attach an elastic component to an absorbent garment. In another embodiment, the method of the present invention may be used to attach a liner to an absorbent structure, attach an outer cover to an absorbent structure, or attach a liner to an outer cover. In still another embodiment, the liner and/or the outer cover may comprise laminates that are formed according to the present invention. Likewise, multiple components in the absorbent core of the article may be adhered on to another or to other components in the absorbent article using the adhesive delivery system of the present invention.

Other features and aspects of the present invention are discussed in greater detail below.

### BRIEF DESCRIPTION OF THE DRAWINGS

A full and enabling disclosure of the present invention, including the best mode thereof to one skilled in the art, is set forth more particularly in the remainder of the specification, including reference to the accompanying figures, in which:

FIG. 1 is a perspective view of one embodiment of a process for adhesively attaching together two components during the construction of a disposable absorbent product;

FIG. 2 is a perspective view of one embodiment of an adhesive nozzle that may be used in accordance with the present invention;

FIG. 3 is a perspective view of another embodiment of a process for applying an adhesive in accordance with the present invention in the formation of disposable absorbent products;

FIG. 4 is a perspective view of one embodiment of a disposable absorbent product made in accordance with the present invention;

FIG. 5 is a perspective view of still another embodiment of a process for applying adhesives in accordance with the present invention;

FIG. 6 is a perspective view of another embodiment of a process for applying adhesives in accordance with the present invention; and

FIG. 7 is a perspective view of still another embodiment of a process for applying adhesives in accordance with the present invention.

Repeat use of reference characters in the present specification and drawings is intended to indicate the same or analogous features or elements of the invention.

### DETAILED DESCRIPTION OF THE INVENTION

It is to be understood by one of ordinary skill in the art that the present discussion is a description of exemplary embodiments only, and is not intended as limiting the broader aspects of the present invention.

The present invention is generally directed to a system and process for applying adhesives in between two components in the formation of disposable absorbent products. The disposable absorbent products may be, for instance, diapers, training pants, swim undergarments, sanitary napkins, adult incontinence products, surgical drapes, wound dressings, and the like. Cleaning articles with absorbent components are also contemplated, such as dry or premoistened wipes comprising two or more adhesively joined components, such as a tissue layer joined to a nonwoven web, as disclosed in commonly owned U.S. application Ser. No. 10/321,277, “Disposable Scrubby Product,” filed Dec. 17, 2002 by Chen et al., and in commonly owned U.S. application Ser. No. 10/036,736, “Sponge-Like Pad Comprising Paper Layers and Method Of Manufacture,” filed Dec. 21, 2001 by Chen et al., both of which are herein incorporated by reference. Cleaning products can also comprise two or more layers of tissue joined to one another, or at least one tissue layer joined to at least one nonwoven web such as spunbond or meltblown web, or two or more nonwoven webs joined together, or a layer of foam such as a melamine-based foam or urethane foam joined to a tissue layer or nonwoven web. Such products may be suitable as mopping wipes, as dish washing wipes, as sponge substitutes, as disposable scrubbing pads, as polishing wipes, as premoistened wipes, and the like.

According to the present invention, adhesive from a nozzle is applied in between two components of a disposable absorbent product (e.g., applied to a surface of at least one of the two components before they are joined together) in which the pattern by which the adhesive is applied and/or the amount per area of adhesive that is applied is varied as a function of distance. By varying the adhesive pattern and/or the amount of adhesive that is applied per area, controlled amounts of adhesive may be applied to the components in order to improve the overall properties of the product. For instance, the adhesive pattern and/or dose may be varied in direct relation to the amount of mechanical stress that the components may be subjected to during use of the product. Further, by controlling adhesive patterns and/or dose, the amount of adhesive used during formation of the product is minimized while retaining all of the above benefits. Generally, the nozzle does not contact the surface to which adhesive is being applied, but is separate from the surface by a finite distance, such as about 1 millimeter (mm) or greater, or about 2 mm or greater, or about 5 mm or greater, such as from about 1.5 mm to about 35 mm, or from about 3 mm to about 15 mm.

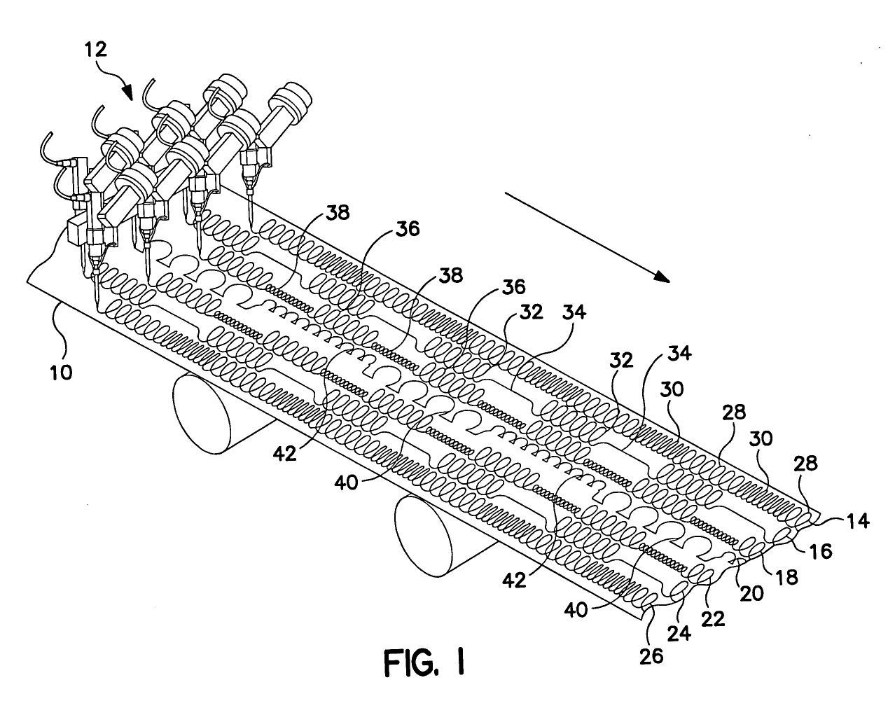

For exemplary purposes, referring to FIG. 1, a substrate 10 is shown in which multiple rows or columns of adhesive are being applied to the substrate in accordance with the present invention. The substrate 10 can be any suitable component that may be used in the formation of an absorbent product. For example, substrate 10 can be an elastic component, a cover material, a liner, an absorbent structure, a multi-layered laminate that may be stretchable or non-stretchable, and the like.

The adhesive being applied to the substrate 10 can also be any suitable adhesive for use in the construction of a disposable absorbent product. The adhesive can be, for instance, a hot-melt adhesive, a pressure sensitive adhesive, a two-component adhesive such as an epoxy, an aqueous or organic solution or dispersion, a UV curable adhesive, and the like.

As illustrated in FIG. 1, the columns or rows of adhesive are emitted onto the substrate 10 by a plurality of nozzles 12. In accordance with the present invention, each column or row of adhesive contains an adhesive pattern that changes as a function of distance. In addition to the adhesive pattern changing as a function of distance, the amount of adhesive applied per unit area may also change. It should be understood, however, that the pattern of the adhesive may change while the amount of adhesive being applied per unit area remains constant.

As shown in FIG. 1, substrate 10 in this embodiment includes seven rows of adhesive 14, 16, 18, 20, 22, 24 and 26. For exemplary purposes, the pattern of adhesive changes as a function of distance over each of the seven rows. For instance, the adhesive pattern in rows 14 and 26 include a first swirl-like portion 28 intermingled with a second swirl-like portion 30. The swirl-like portions 28 and 30 are both formed from a repeating pattern of loops. The density of the loops in the second portion 30, however, is greater than the density of the loops in the first portion 28. In this manner, if desired, greater amounts of adhesive may be applied according to the second portion 30 in comparison to the first portion 28.

In other embodiments, however, the amount of adhesive applied per unit area may remain constant. In this embodiment, the adhesive is spread out in a greater area over the second portion 30. In other words, the amount of adhesive applied according to the first portion 28 of the pattern is the same amount per unit distance as the amount of adhesive that is applied over the second portion 30 of the pattern; however, the adhesive applied according to the second portion 30 covers more surface area than the adhesive applied according to the first portion 28.

The adhesive pattern according to rows 14 and 26 alternates between the pattern of the first portion 28 and the pattern of the second portion 30. The patterns also alternate uniformly in the embodiment shown in FIG. 1. It should be understood, however, that the patterns may alternate in a non-uniform manner depending upon the structural demands of the product being formed.

Rows 16 and 24 on substrate 10 illustrate another embodiment of an adhesive pattern in accordance with the present invention that varies as a function of distance. In rows 16 and 24, the continuous adhesive bead alternates between the pattern of a first portion 32 and the pattern of a second portion 34. The first portion pattern 32 comprises a swirl-like pattern comprised of multiple loops. The pattern of the second portion 34, on the other hand, comprises a linear bead of adhesive.

The linear bead of the second portion 34 is shown in a substantially straight line. It should be understood, however, that various other patterns may be incorporated into the bead of adhesive. For instance, the adhesive nozzle may be controlled in a manner that forms zigzags, sawtooth patterns, scalloped patterns, sinewave patterns, and related patterns such as those provided by commercial sewing machines. In a zigzag pattern or sinewave pattern, for example, the frequency and amplitude of the pattern may vary, as well as the bead size or flow rate of the adhesive to deliver customized adhesive lines tailored for the stresses that portion of the article may encounter.

Referring now to rows 18 and 22, the adhesive pattern in these rows comprises a first portion pattern 36 connected to a second portion pattern 38. In this embodiment, the first portion 36 comprises a large swirl-like pattern, while the second portion 38 comprises a smaller swirl-like pattern.

Referring to row 20, another embodiment of a swirl-like pattern is shown. In this embodiment, the individual swirls are not in the form of loops but in the form of a “omega-like” shape. Further, as shown, the adhesive pattern includes a first portion 40 and a second portion 42. The first portion 40 comprises large swirl-like shapes, while the second portion 42 comprises smaller swirl-like shapes.

As illustrated in FIG. 1, the substrate 10, which can be a web, is conveyed along a conveyor as the adhesive is applied to the substrate using the plurality of nozzles 12. Once the adhesive is applied, the substrate 10 can be adhered to another component in the formation of an absorbent product.

The plurality of rows 14, 16, 18, 20, 22, 24 and 26 in FIG. 1 are provided for exemplary purposes in order to show the various and diverse patterns that may be applied to a substrate in accordance with the present invention. For instance, depending upon the circumstances, more or less rows may be needed on the substrate 10. Further, all of the rows may have the same adhesive bead pattern and may vary at the same location along the distance of the rows.

All of the above described patterns vary with distance and may be used to precisely control adhesive placement as a function of, for instance, the mechanical stresses of a product or for some other functional or aesthetic reason. As the adhesive patterns change, the amount of adhesive applied to the substrate per area may vary and/or the amount of surface area covered by the adhesive may vary. For many embodiments, for instance, the amount of adhesive applied to the substrate in the formation of an absorbent garment may vary from about 1 gsm to about 500 gsm, such as from about 2 gsm to about 50 gsm. When the adhesive bead pattern changes in a manner that changes the adhesive dose, the amount of adhesive applied to the substrate may increase or decrease by at least 10%, at least 20%, at least 40%, at least 50%, at least 60%, at least 70%, or even by greater percentages.

The difference in surface area coverage may also vary widely depending upon the type of product being produced, the type of adhesive being applied to the substrate, and various other factors. In various embodiments, for instance, as the adhesive bead pattern changes as a function of distance, the amount of surface area coverage may change by at least 10%, at least 20%, at least 40%, at least 60%, at least 70%, at least 80%, and by even greater percentages. For example, in some embodiments, such as when going from a swirl-like pattern to a linear bead pattern, the surface area coverage may change by amounts greater than 100%, such as greater than 200%, or even greater than 400%.

The applicator or nozzle used to apply adhesives in accordance with the present invention may vary depending upon the particular application. In general, any suitable adhesive applicator may be used that is capable of dynamically adjusting an adhesive bead being emitted by the applicator. For example, in one embodiment, the PROGRAM-A-SWIRL applicator of Sealant Equipment and Engineering, Inc. of Plymouth, Mich. may be used. The PROGRAM-A-SWIRL applicator is capable of dispensing single and multiple-component adhesives in a pattern that can be rapidly adjusted to vary between a swirl-like pattern and, for instance, a linear bead. In addition to nozzles that are capable of dynamically changing an adhesive pattern, the nozzles can also be placed in operative association with robotic devices that are capable of adjusting the position of a nozzle as a function of time or position. For instance, the height or orientation of a nozzle can be robotically adjusted in order to adjust the breadth or other properties of an adhesive pattern being applied to a substrate as a function of time or position. Dynamic control adhesive application can also be achieved by adjusting the flow of air or other fluids other than the adhesive material associated with operation of an adhesive nozzle. For example, adhesive applied with a meltblown technique can be adjusted by changing the flow of the associated air jets, such as by introducing pulsations in the air flow from acoustic coupling, standing sonic or ultrasonic and other rapid pressure fluctuations that can affect the delivery of the associated adhesive.

In some applications, adhesives are delivered to a nozzle by pumps such as positive displacement pumps which deliver a substantially constant flow of the adhesive to the nozzle, or which maintain a substantially constant pressure of adhesive upstream of the nozzle. In some embodiments, it is desirable to avoid introduction of significant pressure pulsations in the adhesive delivery lines. Thus, in one embodiment, control of the adhesive to dynamically adjust pattern breadth or dosage along the length of an absorbent article is not achieved by increasing the temporal variability in pressure of the adhesive upstream of the nozzle. In another embodiment, control of the adhesive is done without adjusting the flow rate of the adhesive delivered to the nozzle. In one embodiment, dynamic variability in the adhesive applied to an article is achieved by mechanically or acoustically driving the nozzle such the nozzle vibrates, oscillates, or otherwise moves at a scale and speed effective for modifying the delivery of adhesive to the article. In other embodiments, the flow rate or upstream pressure of the adhesive material can be dynamically varied. In other embodiments, not necessarily mutually exclusive with previously discussed embodiments, adhesive delivery may be dynamically varied by adjusting opening internal nozzle geometry, such as the diameter of an opening in cooperative association with a piezoelectric material wherein dimensions can be rapidly adjusted using an electrical signal coupled with piezoelectric material in a nozzle. The nozzle that delivers the adhesive can, in some embodiments, include an ink-jet nozzle such as a piezoelectrically driven nozzle that delivers droplets of adhesive material to the article. However, ink jet nozzles are unsuitable for the delivery of many adhesives or may be unable to meet other demands of the manufacturing system. Therefore, in some embodiment, the nozzle is not an ink jet nozzle, or is not a printing device. The average or typical peak flow rate of adhesive from the nozzle may be at least 0.2 gram per minute (g/min), at least 3 g/min, at least 30 g/min, at least 200 g/min, or at least 1000 g/min, such as from about 1 g/min to about 500 g/min, or from about 1 g/min to about 100 g/min.

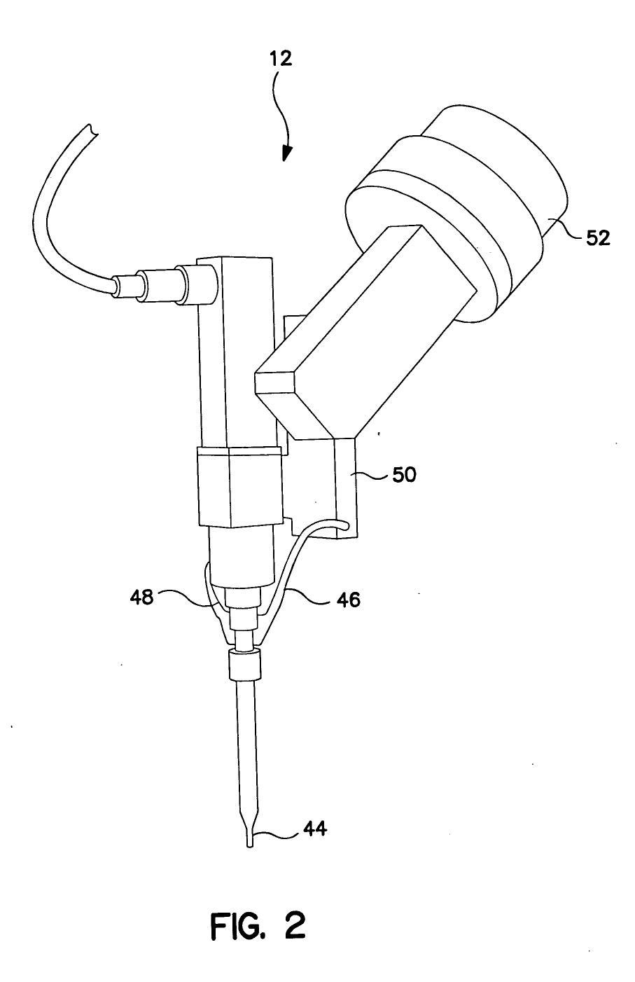

Referring to FIG. 2, one embodiment of a nozzle generally 12 that may be used in accordance with the present invention is shown. As illustrated, the nozzle 12 includes a nozzle tip 44 that is in fluid communication with a first adhesive inlet 46 and a second adhesive inlet 48. The nozzle 12 further includes an oscillating device, such as a servomotor 50. The nozzle 12 is connected to a robotic arm 52 that controls the position of the nozzle tip 44.

The nozzle 12 may be used for single component adhesives or for 2-component reactive adhesives, such as epoxies. When applying a 2-component reactive adhesive, a first component is fed through the inlet 46 while a second component is fed through the inlet 48. The nozzle 12 may include an inline static mixer that blends the two components together prior to exiting the nozzle tip 44. When applying a single component adhesive, on the other hand, the adhesive may be fed through both inlets 46 and 48 or may be fed through a single inlet.

Adhesive is dispensed through the nozzle tip 44 under relatively high pressure. If desired, during application of an adhesive, the upper body of the nozzle may be oscillated by the servomotor 50. For example, in one embodiment, a gimbal in a gear associated with an eccentric device and a bearing oscillates the nozzle tip 44 causing the adhesive to be emitted in a swirl-like pattern. The nozzle may be oscillated at a frequency of greater than about 1,000 rpm, such as greater than about 5,000 rpm. For instance, in one embodiment when producing a relatively high density swirl pattern, the nozzle may be oscillated at a frequency of from about 10,000 rpm to about 20,000 rpm, such as from about 14,000 rpm to about 16,000 rpm. The interaction of the vibration of the nozzle with the adhesive flow from the nozzle tip 44 to a substrate moving below the nozzle tip results in a significant and reproducible amplification of the oscillation into a swirl-like pattern, such as the patterns shown in FIG. 1.

By deactivating the oscillating device or servomotor 50, the adhesive pattern can instantaneously change from a swirl-like pattern to a linear bead of adhesive. The amount of adhesive applied to the substrate can be increased or decreased by increasing or decreasing the amount of pressure under which the adhesive is emitted. Further, the size of the adhesive pattern may be increased or decreased by increasing or decreasing the distance between the nozzle tip 44 and the substrate. Increasing or decreasing the distance between the nozzle tip and the substrate is controlled by controlling the robotic arm 52. For example, increasing the distance between the nozzle tip 44 and a substrate positioned below the nozzle tip increases the size of the swirl-like pattern.

In this regard, the size of the swirl-like pattern can be varied dramatically “on-the-fly” by using the robotic arm 52. For instance, when applying the adhesive according to a plurality of loops, the loops may have a width that varies from about 10 millimeters to about 5 centimeters, such as from about 20 millimeters to about 2 centimeters. To create these patterns, the nozzle tip 44 may be spaced, in one embodiment, from about 1 millimeter to about 5 centimeters from the substrate, such as from about 1 millimeter to about 2 centimeters from the substrate.

The distance between the nozzle tip 44 and the substrate may also be varied by dynamically raising and lowering the substrate, as with a three-dimensional carrier belt (not shown).

When applying a heated material, such as a hot-melt adhesive, various parts of the nozzle 12 may need to be insulated. For example, the oscillating device 50 may need to be insulated from the nozzle to prevent the device from overheating. Further, the nozzle tip 44 may also need to be insulated to prevent the adhesive from cooling and fouling the nozzle tip, or associated with heated air flows around the nozzle.

It should be understood that the nozzle 12 as shown in FIG. 2 represents merely one embodiment of a suitable nozzle that may be used in accordance with the present invention for applying adhesives during the formation of disposable absorbent products. In general, any suitable nozzle may be used that is capable of varying the adhesive pattern instantaneously. For example, in other embodiments, instead of using a servomotor to vibrate the nozzle body, other oscillating devices may be used including piezoelectric devices. Piezoelectric devices can vibrate a nozzle body without the use of a rotating motor. Piezoelectric devices may also be advantageously coupled directly to the nozzle body, which may eliminate the need for an elongated nozzle tip.

Through the use of a nozzle, such as shown in FIG. 2, adhesives may be applied to a substrate according to a controlled pattern that varies as a function of distance. For instance, in accordance with the present invention, an adhesive is delivered to a component of an absorbent product such that the swirl-like pattern, swirl breadth, adhesive dose, etc. may be modified for optimizing an adhesive placement coordinated with and in response to the structural demands of the product being produced. For example, according to the present invention, the adhesive pattern may be increased in size and/or the adhesive dose may be increased at locations on a component where mechanical stresses are at a maximum during use of the product.

The absorbent products that may be formed in accordance with the present invention include diapers, training pants, swim pants, other disposable garments, feminine care products, adult incontinence products, surgical drapes, wound dressings, cleaning products such as multi-component wipes, and the like. For exemplary purposes and in order to better explain the present invention, FIG. 4 depicts one embodiment of a pant-like absorbent article generally 60 that may be constructed using adhesive patterns as described herein.

The article 60 includes a chassis 62 defining a front region 64, a back region 66, and a crotch region 68 interconnecting the front and back regions. The chassis 62 includes a bodyside liner 70 which is configured to contact the wearer, and an outer cover 72 opposite the bodyside liner which is configured to contact the wearer's clothing. An absorbent structure 74 (shown in phantom) is positioned or located between the outer cover 72 and the bodyside liner 70. The absorbent article 60 shown in FIG. 4 has permanently bonded sides. In other embodiments, however, the sides may be refastenable using a suitable attachment structure, such as hook and loop type fasteners. The absorbent article 60 defines a 3-dimensional pant configuration having a waist opening 76 and a pair of leg openings 78. The front region 64 includes the portion of the article 60 which, when worn, is positioned on the front of the wearer while the back region 66 includes the portion of the article which, when worn, is positioned on the back of the wearer. The crotch region 68 of the absorbent article 60 includes the portion of the article which, when worn, is positioned between the legs of the wearer and covers the lower torso of the wearer.

As shown in further detail in FIG. 4, the chassis 62 also defines a pair of longitudinally opposed waist edges which are designated front waist edge 80 and back waist edge 82. The front region 64 is contiguous with the front waist edge 80, and the back region 66 is contiguous with the back waist edge 82. The waist edges 80, 82 are configured to encircle the waist of the wearer when worn and define the waist opening 76.

The illustrated absorbent chassis 62 includes a pair of transversely opposed front side panels 88, and a pair of transversely opposed back side panels 90. The side panels 88, 90 may be integrally formed with the outer cover 72 and/or the bodyside liner 70 or may include two or more separate elements.

The side panels 88 and 90 suitably include an elastic material capable of stretching in a direction generally parallel to the transverse axis of the absorbent article 60. Suitable elastic materials, as well as processes of incorporating side panels into a training pant, are known to those skilled in the art, and are described, for example, in U.S. Pat. No. 4,940,464 issued Jul. 10, 1990 to Van Gompel et al., which is incorporated herein by reference.

The transversely opposed front side panels 88 and transversely opposed back side panels 90 can be permanently bonded to the composite structure comprising the absorbent chassis 62 in the respective front and back regions 64 and 66. Additionally, the side panels 88 and 90 can be permanently bonded to one another using an adhesive in accordance with the present invention.

Each of the side panels 88 and 90 can include one or more individual, distinct pieces of material. In particular embodiments, for example, each side panel 88 and 90 can include first and second side panel portions that are joined at a seam, with at least one of the portions including an elastomeric material. Still alternatively, each individual side panel 88 and 90 can include a single piece of material which is folded over upon itself along an intermediate fold line (not shown). Suitably, the side panels 88 and 90 include an elastic material capable of stretching in a direction generally parallel to the transverse axis of the absorbent article 60.

To enhance containment and/or absorption of body exudates, the absorbent article 60 may include a front waist elastic member 102, a rear waist elastic member 104, and leg elastic members 106, as are all known to those skilled in the art. The waist elastic members 102 and 104 can be operatively joined to the outer cover 72 and/or the bodyside liner 70 along the opposite waist edges 80 and 82, and can extend over part or all of the waist edges. The leg elastic members 106 are suitably operatively joined to the outer cover 72 and/or bodyside liner 70 along opposite side edges of the chassis 62 and positioned in the crotch region 68 of the absorbent article 60.

The waist elastic members 102, 104 and the leg elastic members 106 can be formed of any suitable elastic material. As is well known to those skilled in the art, suitable elastic materials include sheets, strands or ribbons of natural rubber, synthetic rubber, or thermoplastic elastomeric polymers. The elastic materials can be stretched and attached to a substrate, attached to a gathered substrate, or attached to a substrate and then elasticized or shrunk, for example with the application of heat; such that elastic constrictive forces are imparted to the substrate. In one particular embodiment, for example, the leg elastic members 106 include a plurality of dry-spun coalesced multifilament spandex elastomeric threads sold under the trade name LYCRA and available from E.I. DuPont de Nemours and Co., Wilmington, Del.

The absorbent article 60 as shown in FIG. 4 can be made from various materials. The outer cover 72 may be made from a material that is substantially liquid and permeable, and can be elastic, stretchable or nonstretchable. The outer cover 72 can be a single layer of liquid and permeable material, or may include a multi-layered laminate structure in which at least one of the layers is liquid and permeable. For instance, the outer cover 72 can include a liquid permeable outer layer and a liquid and permeable inner layer that are suitably joined together by a laminate adhesive.

For example, in one embodiment, the liquid permeable outer layer may be a spunbond polypropylene nonwoven web. The spunbond web may have, for instance, a basis weight of from about 15 gsm to about 25 gsm.

The inner layer, on the other hand, can be both liquid and vapor impermeable, or can be liquid impermeable and vapor permeable. The inner layer is suitably manufactured from a thin plastic film, although other flexible liquid impermeable materials may also be used. The inner layer prevents waste material from wetting articles such as bedsheets and clothing, as well as the wearer and caregiver. A suitable liquid impermeable film may be a polyethylene film having a thickness of about 0.2 mm.

A suitable breathable material that may be used as the inner layer is a microporous polymer film or a nonwoven fabric that has been coated or otherwise treated to impart a desired level of liquid impermeability. Other “non-breathable” elastic films that may be used as the inner layer include films made from block copolymers, such as styrene-ethylene-butylene-styrene or styrene-isoprene-styrene block copolymers.

As described above, the absorbent structure is positioned in between the outer cover and a liquid permeable bodyside liner 70. The bodyside liner 70 is suitably compliant, soft feeling, and non-irritating to the wearer's skin. The bodyside liner 70 can be manufactured from a wide variety of web materials, such as synthetic fibers, natural fibers, a combination of natural and synthetic fibers, porous foams, reticulated foams, apertured plastic films, or the like. Various woven and nonwoven fabrics can be used for the bodyside liner 70. For example, the bodyside liner can be made from a meltblown or spunbonded web of polyolefin fibers. The bodyside liner can also be a bonded-carded web composed of natural and/or synthetic fibers.

A suitable liquid permeable bodyside liner 70 is a nonwoven bicomponent web having a basis weight of about 27 gsm. The nonwoven bicomponent can be a spunbond bicomponent web, or a bonded carded bicomponent web. Suitable bicomponent staple fibers include a polyethylene/polypropylene bicomponent fiber. In this particular embodiment, the polypropylene forms the core and the polyethylene forms the sheath of the fiber. Other fiber orientations, however, are possible.

The material used to form the absorbent structure 74, for example, may include cellulosic fibers (e.g., wood pulp fibers), other natural fibers, synthetic fibers, woven or nonwoven sheets, scrim netting or other stabilizing structures, superabsorbent material, binder materials, surfactants, selected hydrophobic materials, pigments, lotions, odor control agents or the like, as well as combinations thereof. In a particular embodiment, the absorbent web material is a matrix of cellulosic fluff and superabsorbent hydrogel-forming particles. The cellulosic fluff may comprise a blend of wood pulp fluff. One preferred type of fluff is identified with the trade designation CR 1654, available from US Alliance Pulp Mills of Coosa, Ala., USA, and is a bleached, highly absorbent wood pulp containing primarily soft wood fibers. As a general rule, the superabsorbent material is present in the absorbent web in an amount of from about 0 to about 90 weight percent based on total weight of the web. The web may have a density within the range of about 0.1 to about 0.45 grams per cubic centimeter.

Superabsorbent materials are well known in the art and can be selected from natural, synthetic, and modified natural polymers and materials. The superabsorbent materials can be inorganic materials, such as silica gels, or organic compounds, such as crosslinked polymers. Typically, a suberabsorbent material is capable of absorbing at least about 15 times its weight in liquid, and suitably is capable of absorbing more than about 25 times its weight in liquid. Suitable superabsorbent materials are readily available from various suppliers. For example, FAVOR SXM 880 superabsorbent is available from Stockhausen, Inc., of Greensboro, N.C., USA; and Drytech 2035 is available from Dow Chemical Company, of Midland, Mich., USA.

In addition to cellulosic fibers and superabsorbent materials, the absorbent pad structures may also contain adhesive elements and/or synthetic fibers that provide stabilization and attachment when appropriately activated. Additives such as adhesives may be of the same or different aspect from the cellulosic fibers; for example, such additives may be fibrous, particulate, or in liquid form; adhesives may possess either a curable or a heat-set property. Such additives can enhance the integrity of the bulk absorbent structure, and alternatively or additionally may provide adherence between facing layers of the folded structure.

The absorbent materials may be formed into a web structure by employing various conventional methods and techniques. For example, the absorbent web may be formed with a dry-forming technique, an airlaying technique, a carding technique, a meltblown or spunbond technique, a wet-forming technique, a foam-forming technique, or the like, as well as combinations thereof. Layered and/or laminated structures may also be suitable. Methods and apparatus for carrying out such techniques are well known in the art.

The absorbent web material may also be a coform material. The term “coform material” generally refers to composite materials comprising a mixture or stabilized matrix of thermoplastic fibers and a second non-thermoplastic material. As an example, coform materials may be made by a process in which at least one meltblown die head is arranged near a chute through which other materials are added to the web while it is forming. Such other materials may include, but are not limited to, fibrous organic materials such as woody or non-woody pulp such as cotton, rayon, recycled paper, pulp fluff and also superabsorbent particles or fibers, inorganic absorbent materials, treated polymeric staple fibers and the like. Any of a variety of synthetic polymers may be utilized as the melt-spun component of the coform material. For instance, in some embodiments, thermoplastic polymers can be utilized. Some examples of suitable thermoplastics that can be utilized include polyolefins, such as polyethylene, polypropylene, polybutylene and the like; polyamides; and polyesters. In one embodiment, the thermoplastic polymer is polypropylene. Some examples of such coform materials are disclosed in U.S. Pat. No. 4,100,324 to Anderson, et al.; U.S. Pat. No. 5,284,703 to Everhart, et al.; and U.S. Pat. No. 5,350,624 to Georger, et al.; which are incorporated herein in their entirety by reference for all purposes.

It is also contemplated that elastomeric absorbent web structures may be used. For example, an elastomeric coform absorbent structure having from about 35% to about 65% by weight of a wettable staple fiber, and greater than about 35% to about 65% by weight of an elastomeric thermoplastic fiber may be used to define absorbent pad structures according to the invention. Examples of such elastomeric coform materials are provided in U.S. Pat. No. 5,645,542, incorporated herein in its entirety for all purposes. As another example, a suitable absorbent elastic nonwoven material may include a matrix of thermoplastic elastomeric nonwoven filaments present in an amount of about 3 to less than about 20% by weight of the material, with the matrix including a plurality of absorbent fibers and a super-absorbent material each constituting about 20-77% by weight of the material. U.S. Pat. No. 6,362,389 describes such a nonwoven material and is incorporated herein by reference in its entirety for all purposes. Absorbent elastic nonwoven materials are useful in a wide variety of personal care articles where softness and conformability, as well as absorbency and elasticity, are important.

The absorbent web may also be a nonwoven web comprising synthetic fibers. The web may include additional natural fibers and/or superabsorbent material. The web may have a density in the range of about 0.1 to about 0.45 grams per cubic centimeter. The absorbent web can alternatively be a foam.

In general, any two components of the absorbent garment 60 as shown in FIG. 4 may be adhesively attached together using an adhesive pattern in accordance with the present invention. Using an adhesive pattern that changes as a function of distance allows for the product to be engineered to resist the mechanical stresses placed upon the product in use, which are rarely uniform and can vary significantly with position in the article. Particular examples of components of the absorbent article 60 that may be attached to the article in accordance with the present invention include attaching the front side panels to the garment, attaching the back side panels to the garment, and attaching the front side panels to the back side panels. Adhesive patterns according to the present invention may also be used to attach the front waist elastic members, the rear waist elastic members, and the leg elastic members. In still other embodiments, adhesive patterns may be used in order to attach the liner to the outer cover, the outer cover to the absorbent structure, or the absorbent structure to the liner. Further, adhesive patterns according to the present invention are also well suited to creating a liner, an outer cover, or an absorbent structure that is formed from multiple pieces, such as laminates.

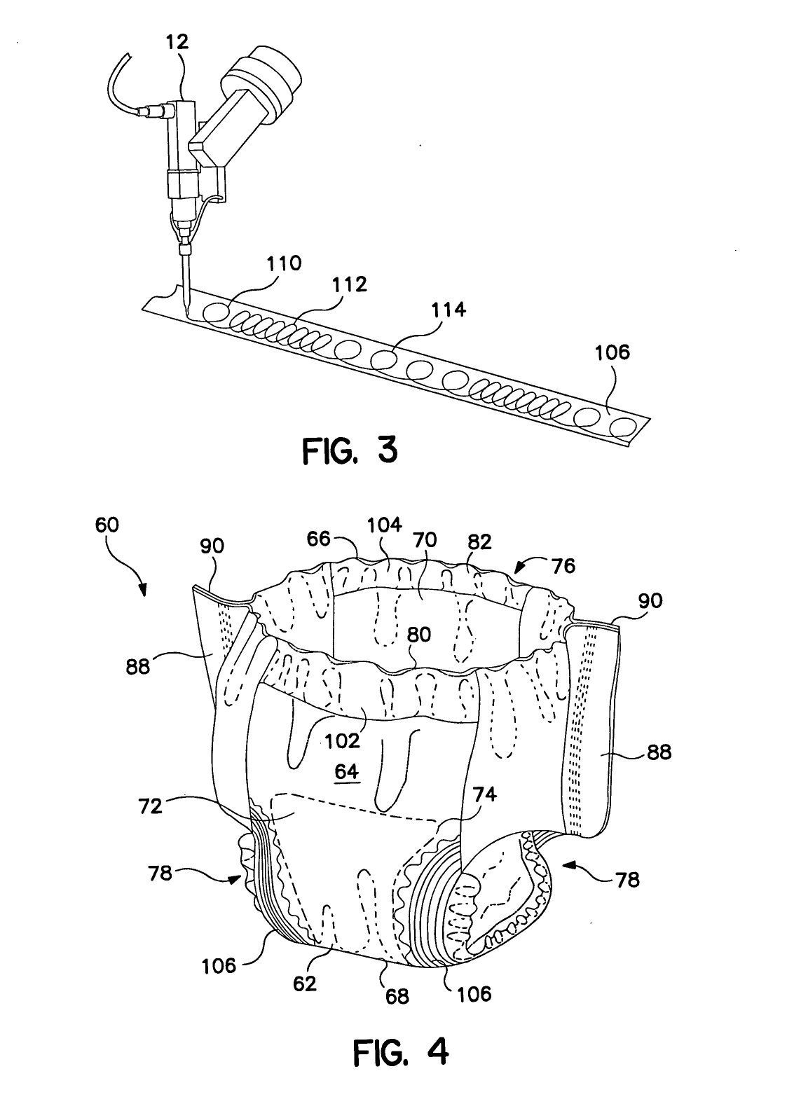

Referring to FIG. 3, for example, a leg elastic member 106 is shown that may be incorporated into the absorbent garment 60 as shown in FIG. 4. In order to attach the leg elastic 106 to the article, an adhesive bead 110 is applied to the leg elastic 106 using the nozzle 12.

In this embodiment, the adhesive pattern 110 includes a first portion 112 comprised of high density loops and a second portion 114 comprised of lower density loops.

When attaching elastic materials, such as the leg elastic 106 to an absorbent product, a careful balance is desired in many applications between firmly attaching the elastic material to the product while at the same time allowing the elastic material to stretch and contract in a comfortable manner when worn. In this regard, in one embodiment of the present invention, the elastic member 106 may be attached to a product using the adhesive bead pattern as shown in FIG. 3. The adhesive bead pattern provides high density areas for firmly attaching the elastic member while also containing low density areas for allowing the elastic member to easily stretch and contract. Further, it should be pointed out that in many applications it is desirable to have a continuous bead of adhesive as opposed to having the adhesive bead be discontinuous and containing gaps where no adhesive is applied, which can affect the performance of the product and the aesthetic look of the product.

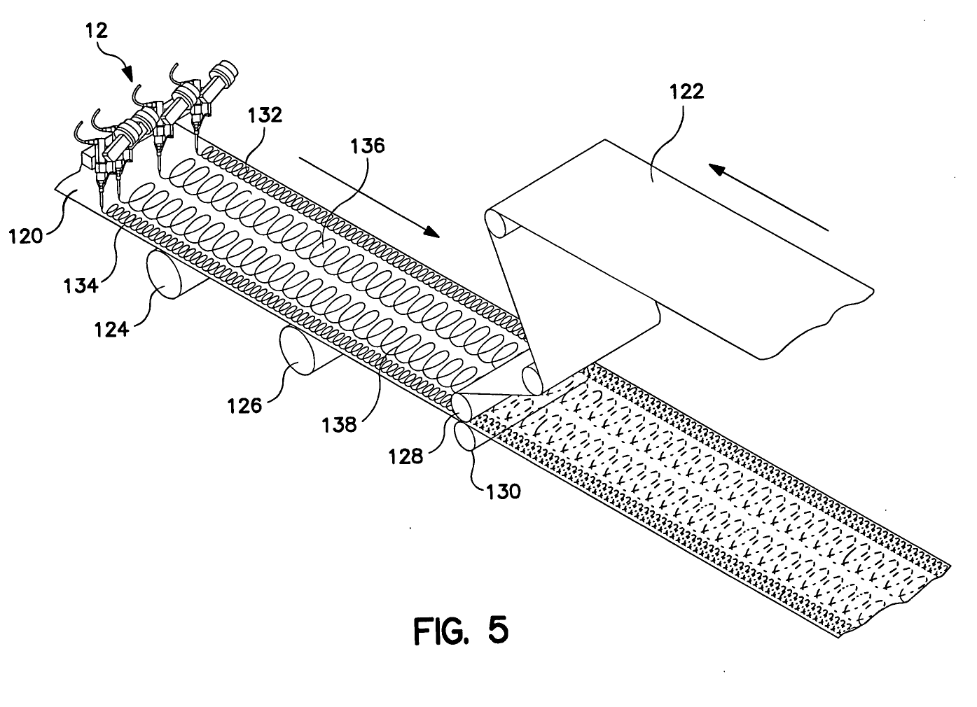

Referring to FIG. 5, still another embodiment of an application of the process of the present invention is illustrated. In FIG. 5, a plurality of nozzles 12 are shown dispensing columns of adhesive in between a first component 20 and a second component 122. In this embodiment, the first component 20 is carried by rollers 124 and 126 below the nozzles 12. The nozzles 12 apply continuous adhesive beads to the first component 120.

After the adhesive is applied to the first component 120, the first component 120 is fed between a pair of nip rollers 128 and 130 for attachment to the second component 122.

In this embodiment, for instance, the first component 120 may be a liner material or an absorbent structure, while the second component 122 may be a cover material. In other embodiments, the first component 120 and the second component 122 may be laminated together in order to form a cover material or a liner.

As shown, in accordance with the present invention, the nozzles 12 apply outside adhesive bead patterns 132 and 134 to the first component and a pair of inner bead patterns 136 and 138 to the first component. All of the bead patterns have a swirl-like pattern. The outer bead patterns 132 and 134 applied along the edges of the material, however, have a much dense pattern and apply greater amounts of adhesive. In this manner, a heavier application of adhesive is applied near the edges of the components for better securing the two components together.

If desired, in an alternative embodiment, each of the adhesive patterns 132, 134, 136 and 138 may also change as a function of distance depending upon the particular application.

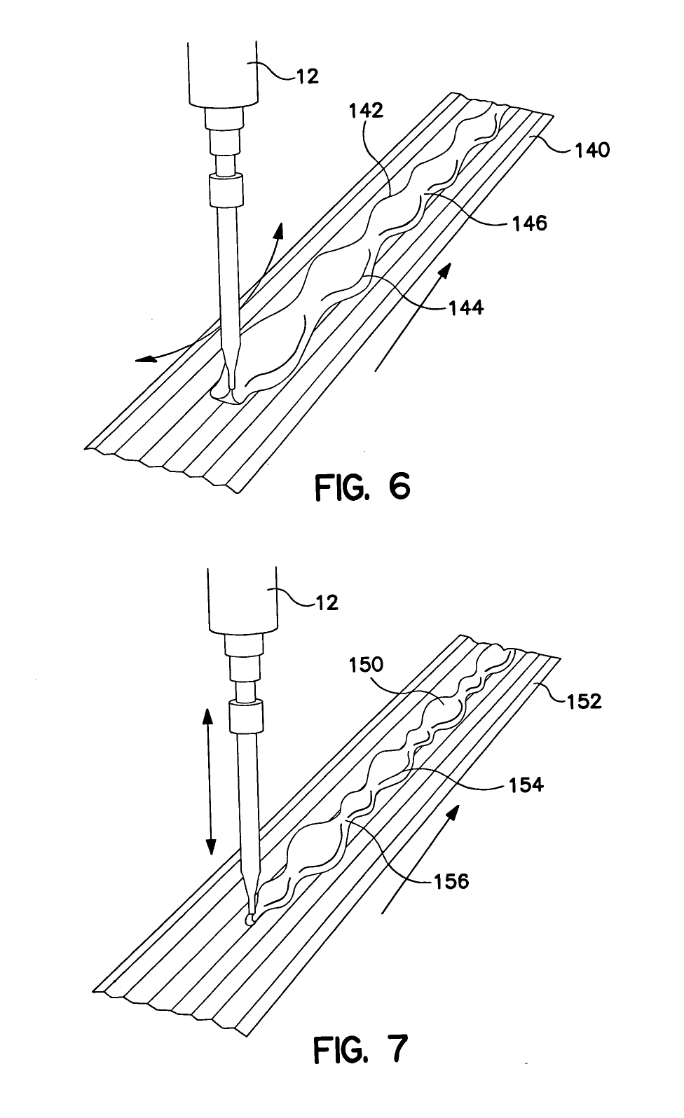

Referring to FIGS. 6 and 7, alternative embodiments of nozzle configurations that may be used in applying adhesives according to the present invention are shown. In particular, the embodiments in FIGS. 6 and 7 relate to methods for modifying traditional nozzles so that adhesive bead patterns may be formed in accordance with the present invention.

For instance, referring to FIG. 6, an adhesive nozzle 12 is shown applying an adhesive bead 142 to a substrate 140. The adhesive pattern is varied as a function of distance by oscillating the nozzle 12 in the machine direction axis as shown by the arrows. In this manner, the nozzle tip alternately moves in the direction of the moving substrate to deliver a higher-than-average dose of adhesive due to a higher dwell time of a portion of the substrate under the nozzle, then moving opposite the direction of the moving substrate to deliver a lower-than-average dose of the adhesive to the substrate due to a lower dwell time at a different portion of the substrate. Thus, the adhesive bead 142 includes an alternating pattern of high dose adhesive areas 144 and low dose adhesive areas 146. The difference in adhesive dose (in terms of weight per area) between the areas 144 and 146 may vary dramatically depending upon the particular application. For example, the difference may be greater than 10%, greater than 25%, greater than 50%, greater than 75%, or even greater than 100%. As shown, however, a continuous bead of adhesive is formed.

In FIG. 7, on the other hand, the adhesive nozzle 12 oscillates periodically up and down in applying a bead of adhesive 150 to a substrate 152. In this manner, the application area of the adhesive periodically increases and decreases. For example, as shown, the adhesive bead 150 includes high coverage areas 154 and low coverage areas 156. In this embodiment, the amount of surface area covered by the adhesive increases and decreases. Depending upon the distance the nozzle 12 is moved up and down, the surface area coverage between the areas 154 and the areas 156 may vary by greater than 10%, greater than 25%, greater than 50%, greater than 75%, and even greater than 100%.

In alternative embodiments, instead of moving the nozzle 12 as shown in FIGS. 6 and 7, the substrate 140 or 152 may be moved in relation to the nozzle to provide the same effects. Also, in other embodiments, the nozzle may oscillate in a forward and backward motion while at the same time oscillating in an up and down motion. In still another embodiment, the nozzle may swivel in the cross machine direction or in diagonal direction to also form a swirl-like pattern during formation of the adhesive bead.

These and other modifications and variations to the present invention may be practiced by those of ordinary skill in the art, without departing from the spirit and scope of the present invention, which is more particularly set forth in the appended claims. In addition, it should be understood that aspects of the various embodiments may be interchanged both in whole or in part. Furthermore, those of ordinary skill in the art will appreciate that the foregoing description is by way of example only, and is not intended to limit the invention so further described in such appended claims.

1. An absorbent garment comprising:
a front portion;
a rear portion;
a crotch area positioned between the front portion and the rear portion;
a pair of opposing leg openings located between the crotch area and the front and rear portions; and
an elastic component attached to the absorbent garment by an adhesive, the adhesive being applied to at least one of the elastic component and the absorbent garment according to a non-uniform pattern that varies as a function of distance, the pattern comprising a first portion and a second portion, the adhesive being present in the first portion in a first amount per area, the adhesive being present in the second portion according to a second amount per area, the second amount per area being less than the first amount per area.

2. An absorbent garment as defined in claim 1, wherein the first portion of the pattern has a swirl-like pattern.

3. An absorbent garment as defined in claim 1, wherein the elastic component comprises a leg elastic that surrounds one of the leg openings.

4. An absorbent garment as defined in claim 1, wherein the second portion of the pattern comprises a portion where no adhesive is present.

5. An absorbent garment as defined in claim 1, wherein the second amount per area is at least 20% less than the first amount per area.

6. An absorbent garment as defined in claim 2, wherein the adhesive is applied in the second portion also according to a swirl-like pattern.

7. An absorbent garment as defined in claim 1, wherein adhesive is applied in the second portion according to a continuous bead.

8. An absorbent garment as defined in claim 1, wherein the non-uniform pattern comprises alternating and repeating first portions and second portions.

9. An absorbent garment as defined in claim 1, wherein the adhesive comprises a hot melt adhesive.

10. An absorbent garment as defined in claim 1, wherein at least a portion of the adhesive in a non-uniform pattern has been delivered by a meltblown process.

11. An absorbent garment as defined in claim 1, wherein the garment comprises a diaper, an adult incontinence product, or a swim garment.

12. An absorbent garment as defined in claim 7, wherein the continuous bead includes a zigzag pattern, a sawtooth pattern, a scalloped pattern, or a sinewave pattern.

13. An absorbent garment comprising:
a liner;
an outer cover;
an absorbent structure positioned between the liner and the outer cover; and
an adhesive positioned between at least two of the liner, the outer cover and the absorbent structure, the adhesive being applied at least partly according to a swirl-like pattern, the adhesive pattern changing as a function of distance, the adhesive pattern changing according to at least one of pattern breadth or adhesive dose in weight per area along said direction.

14. An absorbent garment as defined in claim 13, wherein the swirl-like pattern comprises a plurality of loops having a size, the size of the loops changing as a function of distance.

15. An absorbent garment as defined in claim 13, wherein the swirl-like pattern comprises a plurality of loops having a density in loops per distance, the density of the loops changing as a function of distance.

16. An absorbent garment as defined in claim 13, wherein the adhesive is applied in an amount ranging from about 1 gsm to about 100 gsm.

17. An absorbent garment as defined in claim 13, wherein the adhesive pattern alternates between the swirl-like pattern and a continuous bead.

18. An absorbent garment as defined in claim 13, wherein the adhesive comprises a hot melt adhesive.

19. An absorbent garment as defined in claim 13, wherein the adhesive comprises a pressure sensitive adhesive.

20. An absorbent garment as defined in claim 13, wherein the garment comprises a diaper, an adult incontinence product, or a swim garment.

21. An absorbent garment as defined in claim 13, wherein the adhesive dose of the adhesive pattern changes as a function of distance, and wherein the weight per unit area of adhesive applied varies by at least 20% by weight.

22. An absorbent garment as defined in claim 13, wherein the adhesive dose of the adhesive pattern changes as a function of distance, and wherein the weight per unit area of adhesive applied varies by at least 50% by weight.

23. An absorbent garment as defined in claim 13, wherein the adhesive dose of the adhesive pattern changes as a function of distance, and wherein the weight per unit area of adhesive applied varies by at least 90% by weight.

24. An absorbent garment as defined in claim 17, wherein the continuous bead includes a zigzag pattern, a sawtooth pattern, a scalloped pattern, or a sinewave pattern.

25. An absorbent garment comprising:
a liner;
an outer cover;
an absorbent structure positioned between the liner and the outer cover; and
an adhesive positioned between at least two of the liner, the outer cover and the absorbent structure, the adhesive being applied in columns along a lengthwise direction, each of the columns containing an adhesive pattern, each adhesive pattern comprising at least partly a swirl-like pattern, and wherein greater adhesive is applied adjacent lengthwise edges of the materials in relation to the amount of adhesive applied in a middle area of the materials.

26. An absorbent product comprising multiple components, one of the components comprising an outer cover, while another component comprising an absorbent structure, the outer cover including an exterior surface and an interior surface, the absorbent structure being located adjacent the interior surface of the outer cover; and
an adhesive positioned between at least two components of the absorbent product, the adhesive being applied at least partly according to a swirl-like pattern, the adhesive pattern changing as a function of distance, the adhesive pattern changing according to at least one of pattern breadth or adhesive dose in weight per area along the direction.

27. An absorbent product as defined in claim 26, wherein the swirl-like pattern comprises a plurality of loops having a size, the size of the loops changing as a function of distance.

28. An absorbent product as defined in claim 26, wherein the swirl-like pattern comprises a plurality of loops having a density in loops per distance, the density of the loops changing as a function of distance.

29. An absorbent product as defined in claim 26, wherein the adhesive is applied in an amount ranging from about 1 gsm to about 50 gsm.

30. An absorbent product as defined in claim 26, wherein the adhesive pattern alternates between the swirl-like pattern and a continuous bead.

31. An absorbent product as defined in claim 26, wherein the adhesive comprises a hot melt adhesive.

32. An absorbent product as defined in claim 26, wherein the adhesive comprises a pressure sensitive adhesive.

33. An absorbent product as defined in claim 26, wherein the product comprises a diaper, an adult incontinence product, or a swim garment.

34. An absorbent product as defined in claim 26, wherein the adhesive dose of the adhesive pattern changes as a function of distance, and wherein the weight per unit area of adhesive applied varies by at least 20% by weight.

35. An absorbent product as defined in claim 26, wherein the adhesive dose of the adhesive pattern changes as a function of distance, and wherein the weight per unit area of adhesive applied varies by at least 50% by weight.

36. An absorbent product as defined in claim 26, wherein the adhesive dose of the adhesive pattern changes as a function of distance, and wherein the weight per unit area of adhesive applied varies by at least 90% by weight.

37. An absorbent product as defined in claim 30, wherein the continuous bead includes a zigzag pattern, a sawtooth pattern, a scalloped pattern, or a sinewave pattern.

38. An absorbent article comprising two absorbent layers joined together by an adhesive applied in a pattern by a nozzle, the pattern having a first portion and a second portion, the pattern extending generally in a first direction in the plane of the article, the article also having a second direction in the plane of the article normal to the first direction, wherein at least one parameter selected from the dosage of the adhesive per unit length in the first direction and the breadth of the adhesive in the second orthogonal direction varies along the first direction, such that the at least one parameter measured in the first portion of the pattern differs from the corresponding at least one parameter measured in the second portion by at least 20%.

39. The article of claim 38, wherein the article is a cleaning product.

40. The article of claim 39, wherein the cleaning product is a cleaning wipe comprising at least two fibrous webs adhesively joined together.

41. The article of claim 39, wherein the cleaning product comprises a tissue web joined to a nonwoven web.

42. The article of claim 39, wherein the cleaning product comprises two nonwoven webs.

43. The article of claim 39, wherein the cleaning product comprises a layer of foam.
